(19) United States
(12) Patent Application Publication
(10) Pub. No.: US 2002/0192688 A1
(43) Pub. Date: Dec. 19, 2002

(54) Imaging nucleic acid delivery

(76) Inventors: Xiaoming Yang, Baltimore, MD (US); Ergin Atalar, Columbia, MD (US)

Correspondence Address: DIKE, BRONSTEIN, ROBERTS AND CUSHMAN,, INTELLECTUAL PROPERTY PRACTICE GROUP, EDWARDS & ANGELL, LLP., P.O. BOX 9169, BOSTON, MA, 02209, US

(21) Appl. No.: 10/116,709

(22) Filed: Apr. 4, 2002

Related U.S. Application Data
(60) Provisional application No. 60/281,589, filed on Apr. 5, 2001.

(51) Int. Cl. A61K 49/00; A61K 48/00; C12Q 1/68; C12N 15/861

(52) U.S. Cl. 435/6; 424/9.35; 514/44; 435/320.1; 435/456; 424/93.2

(57) ABSTRACT

The invention provides compositions and methods to monitor delivery of nucleic acids (e.g., such as genes) to a target cell. The compositions comprise a nucleic acid delivery vehicle and a contrast agent. Preferably, the contrast agent is suitable for use in magnetic resonance imaging (MRI). The compositions can be used to monitor the efficacy and selectivity of gene delivery. The invention also provides a medical access device for delivering compositions according to the invention to a target tissue. Preferably, the medical access device comprises a perfusion-porous nucleic acid delivery balloon catheter which can be used in an interventional vascular procedure.

### RELATED APPLICATIONS

This application claims priority to U.S. Provisional Application Serial No. 60/281,589, filed Apr. 5, 2001, the entirety of which is incorporated by reference herein.

### FIELD OF THE INVENTION

The invention relates to compositions and methods for monitoring gene delivery to target tissues.

### BACKGROUND OF THE INVENTION

Atherosclerotic cardiovascular disease remains the leading cause of mortality in the United States (see, e.g., American Heart Association, 1999 Heart And Stroke Statistical Update, Dallas, Tex., American Heart Association). Gene therapy is a rapidly expanding field with great potential for the treatment of atherosclerotic cardiovascular diseases. Several genes, such as vascular endothelial growth factor (VEGF), have been shown to be useful for preventing acute thrombosis, blocking post-angioplasty restenosis, and stimulating growth of new blood vessels (angiogenesis) (Nabel, 1995, Circulation 91: 541-548; Isner, 1999, Hosp. Pract. 34: 69-74). However, precise monitoring of gene delivery into and expression from target atherosclerotic plaques is a challenging task.

Recent in vitro studies have shown that gene expression in cell culture can be detected with imaging techniques, such as nuclear imaging (Tjuvajev, et al., 1995, Cancer Res. 55: 6126-61329; Yu, et al., 2000, Nature Medicine 6: 933-937), optical imaging (Contag, et al., 1998, Nat. Med. 4; 245-247; Yang, et al-, 2001, Radiology 219(1): 171-5) and magnetic resonance (MR) imaging (Johnason, et al., 1993, Magn. Reson. Q. 9: 1-30: 13 14; Weissleder, et al., 2000, Nature Medicine 6: 351-354). Generally, delivery of nucleic acids in vivo has relied on forming complexes (e.g., via chemical bonds) between a contrast agent and a nucleic acid molecule (see, e.g., U.S. Pat. No. 6,232,295 B1; U.S. Pat. No. 6,284,220 B1).

### SUMMARY OF THE INVENTION

The invention provides compositions and methods for monitoring nucleic acid delivery to a target cell. In one aspect, the invention provides a composition comprising an admixture of a nucleic acid molecule, such as a nucleic acid delivery vector, and a contrast agent. Preferably, the nucleic acid molecule is provided in a nucleic acid delivery vehicle which is lipid-based, viral-based, or cell-based. More preferably, the vector comprises a gene operably linked to an expression control sequence.

In one aspect, the nucleic acid molecule comprises a sequence encoding a polypeptide for preventing, correcting and/or normalizing an abnormal physiological response, such as a disease. Exemplary polypeptides include, but are not limited to, hirudin, tissue plasminogen activator, an anchored urokinase activator, a tissue inhibitor of metalloproteinase, proliferating cell nuclear antigen, an angiogenic factor, a tumor suppressor, a suicide gene and a neurotransmitter.

The vector may comprise sequences to facilitate its delivery to, or expression in, a target cell. For example, the vector may comprise a marker gene (e.g., encoding a fluorescent protein) and/or an origin of replication for a host cell and/or target cell.

Preferably, the contrast agent is a magnetic resonance imaging contrast agent. In one aspect, the contrast agent comprises iron or gadolinium. However, the composition generally can comprise any type of contrast agent suitable for use in imaging tissues of an organism.

The composition may comprise a plurality of different types of nucleic acid molecules, e.g., molecules encoding different genes. The composition may further comprise an agent such as a drug, an angiogenic factor, a growth factor, a chemotherapeutic agent, a radionuclide, a protein, a polypeptide, a peptide, a viral protein, a lipid, an amphiphile, a nuclease inhibitor, a polymer, a toxin, a cell, and modified forms and combinations thereof.

The invention also provides a medical access device. The device comprises a housing defining a plurality of channels. At least one channel comprises a delivery channel comprising at least one exit port, while at least one other channel comprises at least an inflation channel comprising at least one exit port. The device further comprises a dilation balloon in communication with the at least one exit port of the inflation channel and a delivery balloon in communication with the at least one exit port of the delivery channel. Preferably, the dilation balloon comprises at least one perfusion channel. More preferably, the delivery balloon also comprises a plurality of pores, through which any of the compositions described above may be delivered to a target cell.

In one aspect, the medical access device comprises at least one channel selected from the group consisting of a guidewire channel, a channel for an optical probe, and a channel for an ultrasound probe. Preferably, the device is a catheter, such as an angiographic catheter, an embolization catheter, a perfusion catheter, or delivery catheter.

In another aspect, the invention provides a method for delivering a nucleic acid to a target cell comprising administering a composition as described above to the target cell. In one aspect, the nucleic acid is encapsulated by a viral protein. Suitable target cells include, but are not limited to, heart cells, liver cells, prostate cells, kidney cells, neural cells, thyroid cells, muscle cells, hematopoietic cells, circulating cells, cells of a blood vessel, and neoplastic cells (e.g., such as tumor cells). Preferably, the target cell is part of a multicellular organism.

In another aspect, the method comprises the step of detecting a signal associated with the contrast agent, such as a magnetic resonance signal. Preferably, the method comprises the step of localizing the signal to a location in a multicellular organism. More preferably, the method comprises obtaining an image of at least the location. Localizing the signal to the location indicates the delivery of the nucleic acid molecule to the location. Preferably, the nucleic acid encodes a gene product necessary for preventing, correcting, and/or normalizing an abnormal physiological response by the target cell. In one aspect, the gene encodes a polypeptide selected from the group consisting of hirudin, tissue plasminogen activator, an anchored urokinase activator, a tissue inhibitor of metalloproteinase, proliferating cell nuclear antigen, an angiogenic factor, a tumor suppressor, a suicide gene and a neurotransmitter.

In one aspect, the nucleic acid molecule further comprises a marker gene and the presence of the marker gene in the target cell is determined. In another aspect, the expression of the marker gene is determined.

The invention also provides a method for delivering an agent to a target cell in a lumen of a body vessel or which is accessible through the walls of a body vessel (e.g., such as a blood vessel). In one aspect, the method comprises positioning a medical access device as described above in the lumen in proximity to a target cell. The dilation balloon is inflated to compress the walls of the body vessel; however, fluids can flow past the device because of the at least one perfusion channel in the dilation balloon. A solution comprising the agent (e.g., such as any of the compositions described above) is delivered through the delivery channel to the delivery balloon and from the delivery balloon to at least a portion of an inner wall of the body vessel. In one aspect, the target cell is a cell which is part of the inner wall of the body vessel (e.g., such as an endothelial cell of a blood vessel). However, in another aspect, the cell is part of a tissue being perfused by the body vessel. Delivery of the agent is monitored by detecting a signal associated with the contrast agent, such as a magnetic resonance signal and, preferably, the body vessel is imaged as well. In one aspect, navigation of the device through the body vessel also is monitored (e.g., using an optical probe positioned in a channel of the device). The device may comprise one or more radioopaque markers to facilitate this process.

### BRIEF DESCRIPTION OF THE FIGURES

The objects and features of the invention can be better understood with reference to the following detailed description and accompanying drawings.

FIG. 1 shows a serial frame from the MR fluoroscopy record of intravascular MR-monitored balloon angioplasty, showing the aortic stenosis (arrow) at the beginning of the procedure and complete opening of the stenosis after total balloon inflation. Arrowheads indicate the circle image artifacts produced by the two alloy rings of the balloon portion of a balloon catheter.

FIG. 2 shows adenoviral vectors mixed with different concentrations of Magnevist according to one aspect of the invention.

FIG. 3 is a schematic of a porous-perfusion gene delivery balloon catheter according to one aspect of the invention. The Figure is not to scale. After inflation of the dilation balloon, the mixture of contrast agent and nucleic acid vector is delivered via the delivery channel through the microholes of the delivery balloon and through the ruptured intima, into atherosclerotic plaques. Blood flows, via the perfusion channels of the dilation balloon, into the distal portion of the target vessel.

FIGS. 4A-C show in vivo intravascular MR images of the femoral artery of a pig. Magnevist (6%)/GFP-lentivirus medium is delivered into the arterial wall using a porous gene delivery balloon catheter. GFP=green fluorescent proteins. FIG. 4A: Before Magnevist/GFP-lentiviral delivery, the balloon is inflated with 6% Magnevist. FIG. 4B: During Magnevist/GFP-lentiviral delivery/infusion, the arterial wall is enhanced by the gadolinium that comes from the delivery microholes or channels (arrows on B) of the porous delivery. FIG. 4C: Immediately after terminating the Magnevist/GFP-lentivirus infusion, the arterial wall is enhanced as a ring (arrows). The parameters of imaging were as follows: ECG-gated fast spin echo sequence, TR/TE=150/10, 16 kHz, 6×6 FOV, 256×128 matrix, 3 NEX, 3-mm thickness, CTLMID coil. These images are taken at three-minute intervals. Magnevist/GFP-lentivirus delivery time=15 minutes.

FIG. 5 shows signal intensity versus time curves from a region-of-interest on an intravascular MR image (IVMRI) of the gadolinium/blue dye-enhanced iliac arterial wall of another pig. Gadolinium/blue delivery time=27 minutes.

FIGS. 6A-C show X-ray angiography on a pig. FIG. 6A: The left internal iliac artery is indicated by an arrow. FIG. 6B: The Remedy catheter is positioned in the same artery. Two arrows indicate two metal markers within the balloon portion. FIG. 6C: Surgery to harvest the targeted artery (arrow).

FIG. 7 shows in vivo intravascular MR images of the internal iliac artery of a pig. Magnevist (6%)/trypan-blue medium is delivered into the arterial wall (open arrows) using the Remedy catheter. A 0.014″ MR antenna is seen within the guidewire channel (long arrow) of the catheter, as well as an air bubble (short arrow) within the inflated balloon. During delivery, the arterial wall is enhanced by the gadolinium that comes from the gene infusion channels (arrowheads) on the lateral aspect of the balloon. These images are taken at three-minute intervals. Scale=1 mm.

FIGS. 8A-B show immunohistochemistry of the untransfected artery (FIG. 8A) and the artery transfected with GFP-lentivirus/Magnevist mixture (FIG. 8B). GFPs are detected as brown-colored precipitates that result in color change of the entire arterial wall from blue to brown (shown as dark grains in FIG. 8B). GFPs locate prominently in the intima (arrows). (200×)

### DETAILED DESCRIPTION

The invention provides compositions and methods to monitor delivery of nucleic acids (e.g., such as genes) to a target cell. The compositions comprise a nucleic acid delivery vehicle and a contrast agent. Preferably, the contrast agent is suitable for use in magnetic resonance imaging (MRI). The compositions can be used to monitor the efficacy and selectivity of gene delivery. The invention also provides a medical access device for delivering compositions according to the invention to a target tissue. Preferably, the medical access device comprises a perfusion-porous nucleic acid delivery balloon catheter which can be used in an interventional vascular procedure.

### Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the phrase “admixture of a nucleic acid and contrast agent” refer to nucleic acids and contrast agents which do not form stable chemical associations (e.g., chemical bonds).

As used herein, the term “peptide” refers to a compound of two or more subunit amino acids, amino acid analogs, or peptidomimetics. The subunits may be linked by peptide bonds or by other bonds (e.g., as esters, ethers, and the like).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. If the peptide chain is long (e.g., greater than about 10 amino acids), the peptide is commonly called a polypeptide or a protein. While the term “protein” encompasses the term “polypeptide”, a “polypeptide” may be a less than full-length protein.

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” to an expression control sequence when the expression control sequence controls and regulates the transcription of that DNA sequence.

As used herein, “coding sequence” is a sequence which is transcribed and translated into a polypeptide when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., mammalian) DNA, and even synthetic DNA sequences. A polyadenylation signal and transcription termination sequence will usually be located 3′ to the coding sequence.

As used herein, “signal sequence” can be included before the coding sequence. This sequence encodes a signal peptide, N-terminal to the polypeptide encoded by the coding sequence, that communicates to a cell to direct the polypeptide to the cell surface or to secrete the polypeptide, and this signal peptide is clipped off by the cell before the protein leaves the cell. Signal sequences can be found associated with a variety of proteins native to prokaryotes and eukaryotes.

As used herein, a “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene).

As used herein, a “nucleic acid delivery vector” is a nucleic acid molecule which can transport a polynucleotide of interest into a cell. Preferably, such a vector comprises a coding sequence operably linked to an expression control sequence. However, a polynucleotide sequence of interest may not necessarily comprise a coding sequence. For example, in one aspect a polynucleotide sequence of interest is an aptamer which binds to a target molecule. In another aspect, the sequence of interest is a complementary sequence of a regulatory sequence which binds to a regulatory sequence to inhibit regulation of the regulatory sequence. In still another aspect, the sequence of interest is itself a regulatory sequence (e.g., for titrating out regulatory factors in a cell).

As used herein, a “nucleic acid delivery vehicle” is defined as any molecule or group of molecules or macromolecules that can carry inserted polynucleotides into a host cell (e.g., such as genes or gene fragments, antisense molecules, ribozymes, aptamers, and the like) and which occurs in association with a nucleic acid vector as described above. For example, nucleic acid delivery vehicles include, but are not limited to: viral capsid proteins (e.g., such as adenoviral, retroviral, and AAV capsid proteins), lipid-based formulations (e.g., multilamellar liposomes, and the like), gas-filled microbubbles, fluorocarbon emulsions, and the like.

As used herein, “nucleic acid delivery,” or “nucleic acid transfer,” refers to the introduction of an exogenous polynucleotide (e.g., such as a “transgene”) into a host cell, irrespective of the method used for the introduction. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

As used herein, a “viral vector” refers to a virus or viral particle that comprises a polynucleotide to be delivered into a host cell, either in vivo, ex vivo or in vitro. Examples of viral vectors include, but are not limited to, adenovirus vectors, adeno-associated virus vectors, retroviral vectors, and the like. In aspects where gene transfer is mediated by an adenoviral vector, a vector construct refers to the polynucleotide comprising the adenovirus genome or part thereof, and a selected, non-adenoviral gene, in association with adenoviral capsid proteins.

As used herein, “adenoviral-mediated gene transfer” or “adenoviral transduction” refers to the process by which a gene or nucleic acid sequences are stably transferred into a host cell by virtue of the adenovirus entering the cell. Preferably, the virus is able to replicate and/or integrate and be transcribed within the cell.

As used herein, “adenovirus particles” are individual adenovirus virions comprised of an external capsid and internal nucleic acid material, where the capsid is further comprised of adenovirus envelope proteins. The adenovirus envelope proteins may be modified to comprise a fusion polypeptide which contains a polypeptide ligand covalently attached to the viral protein, e.g., for targeting the adenoviral particle to a particular cell and/or tissue type.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

As used herein, “hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, a polynucleotide or polynucleotide region (or a polypeptide or polypeptide region) which has a certain percentage (for example, 80%, 85%, 90%, or 95%) of “sequence identity” to another sequence means that, when maximally aligned, using software programs routine in the art, that percentage of bases (or amino acids) are the same in comparing the two sequences.

Two DNA sequences are “substaintally homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art.

The term “biologically active fragment”, “biologically active form”, “biologically active equivalent” of and “functional derivative” of a wild-type protein, such as an angiogenic protein, possesses a biological activity that is at least substantially equal (e.g., not significantly different from) the biological activity of the wild type protein as measured using an assay suitable for detecting the activity.

As used herein, “in vivo” nucleic acid delivery, nucleic acid transfer, nucleic acid therapy” and the like, refer to the introduction of a vector comprising an exogenous polynucleotide directly into the body of an organism, such as a human or non-human mammal, whereby the exogenous polynucleotide is introduced to a cell of such organism in vivo.

As used herein, the term “in situ” refers to a type of in vivo nucleic acid delivery in which the nucleic acid is brought into proximity with a target cell (e.g., the nucleic acid is not administered systemically). For example, in situ delivery methods include, but are not limited to, injecting a nucleic acid directly at a site (e.g., into a tissue, such as a tumor or heart muscle), contacting the nucleic acid with cell(s) or tissue through an open surgical field, or delivering the nucleic acid to a site using a medical access device such as a catheter.

As used herein, the term “isolated” means separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart.

As used herein, a “target cell” or “recipient cell” refers to an individual cell or cell which is desired to be, or has been, a recipient of exogenous nucleic acid molecules, polynucleotides and/or proteins. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism. As used herein, a “target cell” is generally distinguished from a “host cell” in that a target cell is one which is found in a tissue, organ, and/or multicellular organism, while as host cell is one which generally grows in suspension or as a layer on a surface of a culture container.

As used herein, a “subject” is a vertebrate, preferably a mammal, more preferably a human. Mammals include, but are not limited to, murines, simians, humans, farm animals, sport animals, and pets.

The terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

As used herein, a “knock-out” of a target gene means an alteration in the sequence of the gene that results in a decrease of function of the target gene, preferably such that target gene expression is undetectable or insignificant. “Knock-out” transgenics can be transgenic animals having a heterozygous knock-out or a bomozygous knock-out of a gene. “Knock-outs” also include conditional knock-outs, where alteration of the target gene can occur upon, for example, by exposure of the animal to a substance that promotes target gene alteration, (e.g., such as by introduction of an enzyme that promotes recombination at the target gene site).

A “knock-in” of a target gene means an alteration in a host cell genome that results in altered expression (e.g., increased (including ectopic) or decreased expression) of the target gene, e.g., by introduction of an additional copy of the target gene, or by operatively inserting a regulatory sequence that provides for enhanced expression of an endogenous copy of the target gene. “Knock-in” transgenics can be transgenic animals having a heterozygous knock-in of a gene or a homozygous knock-in of a gene. “Knock-ins” also encompass conditional knock-ins.

As used herein, a “composition” refers to the combination of an active agent (e.g., such as a nucleic acid vector) with a contrast agent. The composition additionally can comprise a pharmaceutically acceptable carrier or excipient and/or one or more accessory molecules which may be suitable for diagnostic or therapeutic use in vitro or in vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

As used herein, an “effective amount” is an amount sufficient to affect beneficial or desired results, e.g., such as an effective amount of nucleic acid transfer and/or expression, and/or the attainment of a desired therapeutic endpoint. An effective amount can be administered in one or more administrations, applications or dosages. In one aspect, an effective amount of a nucleic acid delivery vector is an amount sufficient to transform/transduce/transfect at least one cell in a population of cells comprising at least two cells.

As used herein, a “therapeutically effective amount” is used herein to mean an amount sufficient to prevent, correct and/or normalize an abnormal physiological response. In one aspect, a “therapeutically effective amount” is an amount sufficient to reduce by at least about 30 percent, more preferably by at least 50 percent, most preferably by at least 90 percent, a clinically significant feature of pathology, such as for example, size of an ischemic region, size of a tumor mass, elevated blood pressure, fever or white cell count, etc.

An “antibody” is any immunoglobulin, including antibodies and fragments thereof, that binds a specific epitope. The term encompasses polyclonal, monoclonal, and chimeric antibodies (e.g., bispecific antibodies). An “antibody combining site” is that structural portion of an antibody molecule comprised of heavy and light chain variable and hypervariable regions that specifically binds antigen. Exemplary antibody molecules are intact immunoglobulin molecules, substantially intact immunoglobulin molecules, and those portions of an immunoglobulin molecule that contains the paratope, including Fab, Fab′, F(ab′)₂ and F(v) portions, which portions are preferred for use in the therapeutic methods described herein.

### Compositions

### Nucleic Acid Delivery Vehicles

In one aspect, the invention provides a composition comprising a contrast agent with a nucleic acid delivery vehicle. Preferably, such a delivery vehicle comprises at least a nucleic acid delivery vector. Preferably, a nucleic acid delivery vector minimally comprises a polynucleotide sequence for insertion into a target cell and an expression control sequence operably linked thereto to control expression of the polynucleotide sequence (e.g., transcription and/or translation) in the cell. Examples include plasmids, phages, autonomously replicating sequences (ARS), centromeres, and other sequences which are able to replicate or be replicated in vitro or in a host cell and/or target cell, or to convey a polynucleotide to a desired location within a target cell.

Expression control sequences include, but are not limited to, promoter sequences to bind RNA polymerase, enhancer sequences or negative regulatory elements to bind to transcriptional activators and repressors, respectively, and/or translation initiation sequences for ribosome binding. For example, a bacterial expression vector can include a promoter such as the lac promoter and for transcription initiation, the Shine-Dalgamo sequence and the start codon AUG (Sambrook, et al., 1989, supra). Similarly, a eukaryotic expression vector preferably includes a heterologous, homologous, or chimeric promoter for RNA polymerase II, a downstream polyadenylation signal, the start codon AUG, and a termination codon for detachment of a ribosome. Expression control sequences may be obtained from naturally occurring genes or may be designed. Designed expression control sequences include, but are not limited to, mutated and/or chimeric expression control sequences or synthetic or cloned consensus sequences. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.).

In order to optimize expression and/or in vitro transcription, it may be necessary to remove, add or alter 5′ and/or 3′ untranslated portions of the vectors to eliminate extra, or alternative translation initiation codons or other sequences that may interfere with, or reduce, expression, either at the level of transcription or translation. Alternatively, consensus ribosome binding sites can be inserted immediately 5′ of the start codon to enhance expression. a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma, adenovirus, herpes virus and other sequences known to control the expression of genes of mammalian cells, and various combinations thereof.

In one aspect, the nucleic acid delivery vector comprises an origin of replication for replicating the vector. Preferably, the origin functions in at least one type of host cell which can be used to generate sufficient numbers of copies of the sequence for use in delivery to a target cell. Suitable origins therefore include, but are not limited to, those which function in bacterial cells (e.g., such as Escherichia sp., Salmonella sp., Proteus sp., Clostridium sp., Klebsiella sp., Bacillus sp., Streptomyces sp., and Pseudomonas sp.), yeast (e.g., such as Saccharamyces sp. or Pichia sp.), insect cells, and mammalian cells. In one preferred aspect, an origin of replication is provided which functions in the target cell into which the nucleic acid delivery vehicle is introduced (e.g., a mammalian cell, such as a human cell). In another aspect, at least two origins of replication are provided, one that functions in a host cell and one that functions in a target cell.

The nucleic acid delivery vector may alternatively, or additionally, comprise sequences to facilitate integration of at least a portion of the nucleic acid deliver vector into a target cell chromosome. For example, the nucleic acid delivery vector may comprise regions of homology to target cell chromosomal DNA. In one aspect, the delivery vector comprises two or more recombination sites which flank a polynucleotide to be introduced into a cell. Preferably, the recombination sites comprise recognition sequences for a recombinase which can function in a target cell. For example, the recognition sequence may be a loxP site (recognized by the Cre recombinase) (see, e.g., Sauer, 1994, Current Opinion in Biotechnology 5: 521-527; U.S. Pat. No. 4,959,317); attB, attP, attL, and attR sequences (recognized by lambda Integrase) (Landy, 1993, Current Opinion in Biotechnology 3: 699-707). The FLP/FRT system from the Saccharomyces cerevisiae 2μ circle plasmid also may be used (Broach, et al., 1982, Cell 29: 227-234).

The vector may additionally comprise a detectable and/or selectable marker to verify that the vector has been successfully introduced in a target cell and/or can be expressed by the target cell. These markers can encode an activity, such as, but not limited to, production of RNA, peptide, or protein, or can provide a binding site for RNA, peptides, proteins, inorganic and organic compounds or compositions and the like. Examples of detectable/selectable markers genes include, but are not limited to: DNA segments that encode products which provide resistance against otherwise toxic compounds (e.g., antibiotics); DNA segments that encode products which are otherwise lacking in the recipient cell (e.g., tRNA genes, auxotrophic markers); DNA segments that encode products which suppress the activity of a gene product; DNA segments that encode products which can be readily identified (e.g., phenotypic markers such as β-galactosidase, a fluorescent protein (GFP, CFP, YFG, BFP, RFP, EGFP, EYFP, EBFP, dsRed, mutated, modified, or enhanced forms thereof, and the like), and cell surface proteins); DNA segments that bind products which are otherwise detrimental to cell survival and/or function; DNA segments that otherwise inhibit the activity of other nucleic acid segments (e.g., antisense oligonucleotides); DNA segments that bind products that modify a substrate (e.g., restriction endonucleases); DNA segments that can be used to isolate or identify a desired molecule (e.g., segments encoding specific protein binding sites); primer sequences; DNA segments, which when absent, directly or indirectly confer resistance or sensitivity to particular compounds; and/or DNA segments that encode products which are toxic in recipient cells.

The marker gene can be used as a marker for conformation of successful gene transfer and/or to isolate cells expressing transferred genes and/or to recover transferred genes from a cell.

Preferably, the polynucleotide being introduced into the cell comprises a gene or gene fragment that encodes a protein to be expressed in the target cell and/or its progeny, either constitutively, or under selected conditions. However, the polynucleotide may also comprise or encode an RNA sequence, antisense molecule, ribozyme, aptamer, triple helix-forming molecule, and the like. Suitable genes which may be introduced into the target cell depend upon the application. In one aspect, a gene is introduced which can correct or normalize (e.g., diminish symptoms of) an abnormal physiological response (e.g., such as a disease). In another aspect, the gene can prevent an abnormal physiological response. In another aspect, the gene can alter the differentiation state of a cell.

In a particularly preferred aspect, a gene is provided which can prevent, correct, or normalize or improve, an abnormal condition including, but not limited to, hypertension, atherogenesis, thrombosis, intimal hyperplasia, restenosis following angioplasty or stent placement, ischemia, neoplastic diseases (e.g. tumors and tumor metastasis), benign tumors, connective tissue disorders (e.g. rheumatoid arthritis, atherosclerosis), ocular angiogenic diseases (e.g. diabetic retinopathy, macular degeneration, corneal graft rejection, neovascular glaucoma), cardiovascular disease, cerebral vascular disease, diabetes-associated disease and immune disorders.

Substantially similar genes of known genes may also be provided, e.g., genes with greater than about 50%, greater than about 70%, greater than about 90%, and preferably, greater than about 95% identity to a known gene. Percent identity can be determined using software programs known in the art, for example those described in Current Protocols In Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

“Conservatively modified variants” of genes also can be provided. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer, et al., 1991, Nucleic Acid Res. 19: 5081; Ohtsuka, et al., 1985, J. Biol. Chem. 260: 2605-2608; Rossolini et al., 1994, Mol. Cell. Probes 8: 91-98).

In another aspect, a substantially similar gene is one which specifically hybridizes to the known gene under stringent hybridization conditions. Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

In some aspects, multiple different types of nucleic acid delivery vectors are provided, e.g., encoding different types of genes which may act together to promote a therapeutic effect, or to increase the efficacy or selectivity of gene transfer and/or gene expression in a cell.

The nucleic acid delivery vector may be provided as naked nucleic acids or in a delivery vehicle associated with one or more molecules for facilitating entry of a nucleic acid into a cell. Suitable delivery vehicles include, but are not limited to: liposomal formulations (e.g., such as multilamellar liposomes), polypeptides; polysaccharides; lipopolysaccharides, viral formulations (e.g., including viruses, viral particles, artificial viral envelopes and the like), gas-filled microbubbles, fluorocarbon emulsions, cell delivery vehicles, and the like.

### Naked Nucleic Acids

The technique of somatic gene therapy using direct DNA injection into myocardium has several advantages compared with other previously described methods of gene therapy. Direct injection of recombinant DNA into the myocardium is useful for the treatment of many acquired and inherited cardiovascular diseases in particular, by stimulating collateral circulation in areas of chronic myocardial ischemia by expressing recombinant angiogenesis factors locally in the ventricular wall. For example, U.S. Pat. No. 6,331,524 describes direct injection of a purified nucleic acid delivery vector into the heart wall of rats and efficient, long-term (at least 6 months) expression of the vector.

It is also possible to deliver a nucleic acid delivery vector directly to the arteries following a surgical operation.

### Lipid-Based Formulations

Delivery vehicles designed to facilitate intracellular delivery of biologically active molecules must interact with both non-polar and polar environments (in or on, for example, the plasma membrane, tissue fluids, compartments within the cell, and the like). Therefore, preferably, delivery vehicles are designed to contain both polar and non-polar domains or a translocating sequence for translocating a nucleic acid into a cell.

Compounds having polar and non-polar domains are termed amphiphiles. Cationic amphiphiles have polar groups that are capable of being positively charged at, or around, physiological pH for interacting with negatively charged polynucleotides such as DNA.

The nucleic acid vectors described above can be provided in formulations comprising lipid monolayers or bilayers to facilitate transfer of the vectors across a cell membrane. Liposomes or any form of lipid membrane, such as planar lipid membranes or the cell membrane of an intact cell, e.g., a red blood cell, can be used. Liposomal formulations can be administered by any means, including administration intravenously or orally.

Liposomes and liposomal formulations can be prepared according to standard methods and are well known in the art, see, e.g., Remington's; Akimaru, 1995, Cytokines Mol. Ther. 1: 197-210; Alving, 1995, Immunol. Rev. 145: 5-31; Szoka, 1980, Ann. Rev. Biophys. Bioeng. 9: 467; U.S. Pat. No. 4,235,871; U.S. Pat. No. 4,501,728; and U.S. Pat. No. 4,837,028. In one aspect, the liposome comprises a targeting molecule for targeting a liposome:nucleic acid vector complex to a particular cell type. In a particularly preferred aspect, a targeting molecule comprises a binding partner (e.g., a ligand or receptor) for a biomolecule (e.g., a receptor or ligand) on the surface of a blood vessel or a cell found in a target tissue (e.g., such as the heart). In one aspect, liposomes comprise a molecule positioned on the surface of the liposome in such a manner that the molecule is available for interaction with the receptors or ligands on endothelial cells. In another aspect, the molecule is a heart homing peptide, as described in U.S. Pat. No. 6,303,573, for example.

Liposome charge is an important determinant in liposome clearance from the blood, with negatively charged liposomes being taken up more rapidly by the reticuloendothelial system (Juliano, 1975, Biochem. Biophys. Res. Commun. 63: 651) and thus having shorter half-lives in the bloodstream. Incorporating pbosphatidylethanolamine derivatives enhances the circulation time by preventing liposomal aggregation. For example, incorporation of N-(omega-carboxy)acylamidophosphatidylethanolamines into large unilamellar vesicles of L-alpha-distearoylphosphatidylcholine dramatically increases the in vivo liposomal circulation lifetime (see, e.g., Ahl, 1997, Biochim. Biophys. Acta 1329: 370-382). Liposomes with prolonged circulation half-lives are typically desirable for therapeutic and diagnostic uses. For a general discussion of pharmacokinetics, see, e.g., Remington's, Chapters 37-39, Lee, et al., In Pharmacokinetic Analysis: A Practical Approach (Technomic Publishing AG, Basel, Switzerland 1996).

Typically, liposomes are prepared with about 5 to 15 mole percent negatively charged phospholipids, such as phosphatidylglycerol, phosphatidylserine or phosphatidyl-inositol. Added negatively charged phospholipids, such as phosphatidylglycerol, also serve to prevent spontaneous liposome aggregation, and thus minimize the risk of undersized liposomal aggregate formation. Membrane-rigidifying agents, such as sphingomyelin or a saturated neutral phospholipid, at a concentration of at least about 50 mole percent, and 5 to 15 mole percent of monosialylganglioside can also impart desirably liposome properties, such as rigidity (see, e.g., U.S. Pat. No. 4,837,028).

Additionally, the liposome suspension can include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

The nucleic acid delivery vehicles of the invention can include multilamellar vesicles of heterogeneous sizes. For example, vesicle-forming lipids can be dissolved in a suitable organic solvent or solvent system and dried under vacuum or an inert gas to form a thin lipid film. If desired, the film can be redissolved in a suitable solvent, such as tertiary butanol, and then lyophilized to form a more homogeneous lipid mixture which is in a more easily hydrated powderlike form. This film is covered with an aqueous solution of the peptide or polypeptide complex and allowed to hydrate, typically over a 15 to 60 minute period with agitation. The size distribution of the resulting multilamellar vesicles can be shifted toward smaller sizes by hydrating the lipids under more vigorous agitation conditions or by adding solubilizing detergents such as deoxycholate. The hydration medium preferably comprises the nucleic acid at a concentration which is desired in the interior volume of the liposomes in the final liposome suspension.

Following liposome preparation, the liposomes can be sized to achieve a desired size range and relatively narrow distribution of liposome sizes. One preferred size range is about 0.2 to 0.4 microns, which allows the liposome suspension to be sterilized by filtration through a conventional filter, typically a 0.22 micron filter. Filter sterilization can be carried out on a high throughput basis if the liposomes have been sized down to about 0.2 to 0.4 microns. Several techniques are available for sizing liposome to a desired size (see, e.g., U.S. Pat. No. 4,737,323).

Suitable lipids include, but are not limited to, DOTMA (Feigner, et al., 1987, Proc. Natl. Acad. Sci. USA 84: 7413-7417), DOGS or Transfectain™ (Behr, et al., 1989, Proc. Natl. Acad. Sci. USA 86: 6982-6986), DNERIE or DORIE (Feigner, et al., Methods 5: 67-75), DC-CHOL (Gao and Huang, 1991, BBRC 179: 280-285), DOTAPTM (McLachlan, et al., 1995, Gene Therapy 2: 674-622), Lipofectamine® and glycerolipid compounds (see, e.g., EP901463 and W098/37916).

Other molecules suitable for complexing with nucleic acid delivery vectors include cationic molecules, such as, polyamidoamine (Haensler and Szoka, 1993, Bioconjugate Chem. 4: 372-379), dendriticpolyiner (WO 95/24221), polyethylene irinine or polypropylene h-nine (WO 96/02655), polylysine (U.S. Pat. No. 5,595,897; FR 2 719 316), chitosan (U. S. Pat. No. 5,744,166) or DEAE dextran (Lopata, et al., 1984, Nucleic Acid Res. 12: 5707-5717).

### Viral-Based Gene Delivery Vehicles

In one aspect, the nucleic acid delivery vehicle comprise a virus or viral particle. In this aspect, preferably, the nucleic acid vector comprises a viral vector. Viral vectors, such as retroviruses, adenoviruses, adeno-associated viruses and herpes viruses, are often made up of two components, a modified viral genome and a coat structure surrounding it (see, e.g., Smith et al., 1995, Ann. Rev. Microbiol. 49: 807-838), although sometimes viral vectors are introduced in naked form or coated with proteins other than viral proteins. Most current vectors have coat structures similar to a wild-type virus. This structure packages and protects the viral nucleic acid and provides the means to bind and enter target cells.

Preferably, viral vectors are modified from wild-type viral genomes to disable the growth of the virus in a target cell while enabling the virus to grow in a host cell (e.g., such as a packaging or helper cell) used to prepare infectious particles. Vector nucleic acids generally essential cis-acting viral sequences for replication and packaging in a helper line and expression control sequences for regulating the expression of a polynucleotide being delivered to a target cell. Other viral functions are expressed in trans in specific packaging or helper cell lines as are known in the art.

Preferred vectors are viral vectors derived from a virus selected from the group consisting of herpes viruses, cytomegaloviruses, foamy viruses, lentiviruses, Semliki forrest virus, AAV (adeno-associated virus), poxvituses, adenovirases and retroviruses. Such viral vectors are well known in the art.

In one preferred aspect, a viral vector used is an adenoviral vector. The adenoviral genome consists of a linear double- stranded DNA molecule of approximately 36 kb carrying more than about thirty genes necessary to complete the viral replication cycle. The early genes are divided into 4 regions (E1 to E4) that are essential for viral replication with the exception of the E3 region, which is believed to modulate the anti-viral host immune response. The E1 region (E1A and E1B) encodes proteins responsible for the regulation of transcription of the viral genome. Expression of the E2 region genes (E2A and E2B) leads to the synthesis of the polypeptides needed for viral replication. The proteins encoded by the E3 region prevent cytolysis by cytotoxic T cells and tumor necrosis factor (Wold and Gooding, 1991, Virology 184: 1-8). The proteins encoded by the E4 region are involved in DNA replication, late gene expression and splicing and host cell shut off (Halbert, et al., 1985, J. Virol. 56: 250-257). The late genes generally encode structural proteins contributing to the viral capsid. In addition, the adenoviral genome carries at cis-acting 5′ and 3′ ITRs (Inverted Terminal Repeat) and packaging sequences essential for DNA replication. The ITRs harbor origins of DNA replication while the encapsidation region is required for the packaging of adenoviral DNA into infectious particles.

Adenoviral vectors can be engineered to be conditionally replicative (CRAd vectors) in order to replicate selectively in specific cells (e.g., such as proliferative cells) as described in Heise and Kim (2000, J. Clin. Invest. 105: 847- 85 1). In another aspect, an adenoviral vector is replication-defective for the E1 function (e.g., by total or partial deletion or mutagenesis of E1). The adenoviral backbone of the vector may comprise additional modifications (deletions, insertions or mutations in one or more viral genes). An example of an E2 modification is illustrated by the thermosensitive mutation localized on the DBP (DNA Binding Protein) encoding gene (Ensinger et al., 1972, J. Virol. 10: 328-339). The adenoviral sequence may also be deleted of all or part of the E4 region (see, e.g., EP 974 668; Christ, et al., 2000, Human Gene Ther. 11: 415-427; Lusky, et al., 1999, J. Virol. 73: 8308-8319). Additional deletions within the non-essential E3 region may allow the size of the polynucleotide being delivered to be increased (Yeb, et al., 1997, FASEB Journal 11: 615 623). However, it may be advantageous to retain all or part of the E3 sequences coding for polypeptides (e.g., such as gpl9k) allowing the virus to escape the immune system (Gooding, et al., 1990, Critical Review of Immunology 10: 53-71) or inflammatory reactions (EP 00440267.3).

Second generation vectors retaining the ITRs and packaging sequences and comprising substantial genetic modifications to abolish the residual synthesis of the viral antigens also may be used in order to improve long-term expression of the expressed gene in the transduced cells (see, e.g., WO 94/28152; Lusky, et al., 1998, J. Virol 72: 2022-2032).

The polynucleotide being introduced into the cell may be inserted in any location of the viral genome, with the exception of the cis-acting sequences. Preferably, it is inserted in replacement of a deleted region (E1, E3 and/or E4), preferably, within a deleted E1 region.

Adenoviruses can be derived from any human or animal source, in particular canine (e.g. CAV-1 or CAV-2 Genbank ref. CAVIGENOM and CAV77082, respectively), avian (Genbank ref. AAVEDSDNA), bovine (such as BAV3; Reddy, et al., 1998, J. Virol. 72:1394 1402), murine (Genbank ref. ADRMUSMAVI), ovine, feline, porcine or simian sources or alternatively, may be a hybrid virus. Any serotype can be employed. However, the human adenoviruses of the C sub-group are preferred, especially adenoviruses 2 (Ad2) and 5 (Ad5). Such viruses are available, for example, from the ATCC.

Adenoviral particles or empty adenoviral capsids also can be used to transfer nucleic acid delivery vectors by a virus-mediated cointernalization process as described in U.S. Pat. No. 5,928,944. This process can be accomplished in the presence of cationic agent(s) such as polycarbenes or lipid vesicles comprising one or more lipid layers.

Adenoviral particles may be prepared and propagated according to any conventional technique in the field of the art (e.g., WO 96/17070) using a complementation cell line or a helper virus, which supplies in trans the missing viral genes necessary for viral replication. The cell lines 293 (Graham et al., 1977, J. Gen. Virol. 36: 59-72) and PERC6 (Fallaux et al., 1998, Human Gene Therapy 2: 1909-1917) are commonly used to complement E1 deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and WO 97/04119). The adenoviral particles can be recovered from the culture supernatant but also from the cells after lysis and optionally further purified according to standard techniques (e.g., chromatography, ultracentrifugation, as described in WO 96/27677, WO 98/00524 WO 98/26048 and WO 00/50573).

The retroviral particles are preferably recovered from the culture supernatant and may optionally be further purified according to standard techniques (e.g. chromatography, ultracentrifugation).

Cell-type specific targeting may be achieved with vectors derived from adenoviruses having a broad host range by the modification of viral surface proteins. For example, the specificity of infection of adenoviruses is determined by the attachment to cellular receptors present at the surface of permissive cells. In this regard, the fiber and penton present at the surface of the adenoviral capsid play a critical role in cellular attachment (Defer, et al., 1990, J. Virol. 64: 3661-3673). Thus, cell targeting of adenoviruses can be carried out by genetic modification of the viral gene encoding fiber and/or penton, to generate modified fiber and/or penton capable of specific interaction with unique cell surface receptors. Examples of such modifications are described in Wickarn, et al., 1997, J. Virol 71: 8221-8229; Arriberg, et al., 1997, Virol Chem 268: 6866-6869; Roux, et al., 1989, Proc. Natl. Acad Sci. USA 86: 9079-9083; Miller and Vile, 1995, FASEB J. 9:190-199; WO 93/09221, and in WO 95/28494.

In other aspects, retroviral vectors are used. Retroviruses are a class of integrative viruses which replicate using a virus-encoded reverse transcriptase, to replicate the viral RNA genome into double stranded DNA which is integrated into chromosomal DNA of the infected cells (e.g., target cells). Such vectors include those derived from murine leukemia viruses, especially Moloney (Gilboa, et al., 1988, Adv. Exp.Med. Biol. 241: 29) or Friend's FB29 strains (WO 95/01447). Generally, a retroviral vector is deleted of all or part of the viral genes gag, pol and env and retains 5′and 3′ LTRs and an encapsidation sequence. These elements may be modified to increase expression level or stability of the retroviral vector. Such modifications include the replacement of the retroviral encapsidation sequence by one of a retrotransposon such as VL30 (see, e.g., U.S Pat. No. 5,747,323). Preferably, the polynucleotide of interest is inserted downstream of the encapsidation sequence, preferably in opposite direction relative to the retroviral genome. Cell specific targeting may be achieved by the conjugation of antibodies or antibody fragments to the retroviral envelope protein as is know in the art.

Retroviral particles are prepared in the presence of a helper virus or in an appropriate complementation (packaging) cell line which contains integrated into its genome the retroviral genes for which the retroviral vector is defective (e.g. gag/pol and env). Such cell lines are described in the prior art (Miller and Rosman, 1989, BioTechniques 7: 980; Danos and Mulligan, 1988, Proc. Natl. Acad. Sci. USA 85: 6460; Markowitz, et al., 1988, Virol. 167: 400). The product of the env gene is responsible for the binding of the viral particle to the viral receptors present on the surface of the target cell and, therefore determines the host range of the retroviral particle. in the context of the invention, it is advantageous to use a packaging cell line, such as the PA317 cells (ATCC CRL 9078) or 293E16 (W097/35996) containing an amphotropic envelope protein, to allow infection of human and other species' target cells.

Other suitable viruses include poxviruses. The genome of several members of poxviridae has been mapped and sequenced. A poxviral vector may be obtained from any member of the poxviridae, in particular canarypox, fowlpox and vaccinia virus. Suitable vaccinia viruses include, but are not limited to, the Copenhagen strain (Goebel, et al., 1990, Virol. 179: 247-266; Johnson, et al., 1993, Virol. 196: 381-401), the Wyeth strain and the modified Ankara (MVA) strain (Antoine, et al., 1998, Virol. 244: 365-396). The general conditions for constructing a vaccinia virus vector are known in the art (see, e.g., EP 83 286 and EP 206 920; Mayr et al., 1975, Infection 3: 6-14; Sutter and Moss, 1992, Proc. Natl. Acad. Sci. USA 89: 10847-10851). Preferably, the polynucleotide of interest is inserted within a non-essential locus such as the nOD7coding intergenic regions or any gene for which inactivation or deletion does not significantly impair viral growth and replication.

Poxviral particles are prepared as described in the art (Piccini, et al., 1987, Methods of Enzymology 153: 545-563; U.S. Pat. No. 4,769,330; U.S. Pat. No. 4,772,848; U.S. Pat. No. 4,603,112; U.S. Pat. No. 5,100,587 and U.S. Pat. No. 5,179,993). Generally, a donor plasmid is constructed, amplified by growth in E. coli and isolated by conventional procedures. Then, it is introduced into a suitable cell culture (e.g. chicken embryo fibroblasts) together with a poxvirus genome, to produce, by homologous recombination, poxviral particles. These can be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentriftigation on cesium chloride or sucrose gradients).

Viral capsid molecules may include targeting moieties to facilitate targeting and/or entry into cells. Suitable targeting molecules, include, but are not limited to: chemical conjugates, lipids, glycolipids, hormones, sugars, polymers (e.g. PEG, polylysine, PEI and the like), peptides, polypeptides (see, e.g., WO 94/40958), vitamins, antigens, lectins, antibodies and fragments thereof. Preferably, such targeting molecules recognize and bind to cell-specific markers, tissue-specific markers, cellular receptors, viral antigens, antigenic epitopes or tumor-associated markers.

A composition based on viral particles may be formulated in the form of doses of between 10 and 10¹⁴ i.u. (infectious units), and preferably, between 10 and 10¹¹ i.u. The titer may be determined by conventional techniques. The doses of nucleic acid delivery vector are preferably comprised between 0.01 and 10 mg/kg, more especially between 0.1 and 2 mg/kg.

### Cell-Based Delivery Vehicles

The nucleic acid vectors according to the invention can be delivered to target cells by means of other cells (“delivery cells) which comprise the vectors. Methods for introducing vectors into cells are known in the art and include microinjection of DNA into the nucleus of a cell (Capechi, et al., 1980, Cell 22: 479-488); transfection with CaP0₄ (Chen and Okayama, 1987, Mol. Cell Biol. 7: 2745 2752), electroporation (Chu, et al., 1987, Nucleic Acid Res. 15: 1311-1326); lipofection/liposome fusion (Feigner, et al., 1987, Proc. Natl. Acad. Sci. USA 84: 7413-7417) and particle bombardment (Yang, et al., 1990, Proc. Natl. Acad. Sci. USA 87: 9568-9572). Suitable cells include autologous and non-autologous cells, and may include xenogenic cells. Delivery cells may be induced to deliver their contents to the target cells by inducing their death (e.g., by providing inducible suicide genes to these cells).

### Contrast Agents

Contrast agents according to the invention generally are those useful in diagnostic imaging methods including, but not limited to: X-ray, x-ray computed tomography (CT) imaging, including CT angiography (CTA) imaging, magnetic resonance (MR) imaging, magnetic resonance angiography (NIA), nuclear medicine, ultrasound (US) imaging, optical imaging, elastography, infrared imaging, microwave imaging, and the like. Preferably, contrast agents are biocompatible (e.g., non-toxic, chemically stable and/or non-reactive with tissues). In one aspect, a contrast agent comprises a limited lifetime before elimination from the body. This lifetime may be longer or shorter than the lifetime of the nucleic acid delivery vector.

For x-ray or computed tomography imaging, the contrast agent should have a different electron density than surrounding tissues (either more or less electron density) to render it visible. With respect to contrast agents for CT, it is generally desirable to employ agents that will increase electron density in certain areas of a region of the body (positive contrast agents). Suitable electron density is achieved, for example, in compounds with bromine, fluorine or iodine moieties, and in materials comprising or including radioopaque metal atoms. It also may be desirably to employ agents that will decrease electron density in certain areas of a region of the body (negative contrast agents).

Ultrasound and x-ray imaging, including the use of digital subtraction techniques, may also be utilized according to one aspect of the present invention. An ultrasound contrast agent can be selected on the basis of density or acoustical properties. Preferably, the contrast agent is echogenic. As employed herein, the term “echogenic” refers to a contrast agent that may be capable of reflecting or emitting sound waves. Echogenic contrast agents may be particularly useful to alter, for example, the acoustic properties of a lymph tissue, organ or region of a patient, preferably the sentinel lymph node, thereby resulting in improved contrast in diagnostic imaging techniques. Suitable contrast agents for use in such applications include, but are not limited to: a microbubble contrast agent, Imagent (AF0150) (Alliance Pharmaceutical Corp., San Diego, Calif.; AI-700); Albunex and Optison (FS069) (Molecular Biosystems, Inc., San Diego, Calif.); Echogen (QW7437) (Sonus Pharmaceuticals, Bothell, Wash.); Levovist (SH/TA-508), Echovist and Sonovist (SHU563), (Schering AG, Berlin, Germany); Aerosomes-DMP115 and MRX115 (ImaRx Pharmaceuticals, Tucson, Ariz.); BR1 and BR14 (Bracco International B.V., Amsterdam, NL), Quantison and Quantison Depot (Andaris, Ltd. Nottingham, GB); and NC 100 (Nycomed Imaging AS, Oslo, Norway), and the like. Contrast agents and methods of forming contrast agents also are disclosed in U.S. Pat. No. 4,957,656; U.S. Pat. No. 5,141,738; U.S. Pat. No. 4,657,756; U.S. Pat. No. 5,558,094; U.S. Pat. No. 5,393,524; U.S. Pat. No. 5, 558,854; U.S. Pat. No. 5,573,751; U.S. Pat. No. 5,558,853; U.S. Pat. No. 5,595,723; U.S. Pat. No. 5,558,855; U.S. Pat. No. 5,409,688; and U.S. Pat. No. 5,567,413.

Preferably, however, a contrast agent is selected which is suitable for MRI. MRI is a diagnostic imaging technique which employs a magnetic field, field gradients and radiofrequency energy to excite protons and make an image of the mobile protons in water and fat (i.e., molecules found in cells).

MRI contrast agents primarily act by affecting T1 or T2 relaxation of water protons (described further below). Most contrast agents generally shorten T1 and/or T2. When contrast agents shorten T1, this increases signal intensity on T1 weighted images. When contrast agents shorten T2, this decreases signal intensity particularly on T2 weighted pulse sequences. Thus, preferably, contrast agents used in the invention have adequate nuclear or relaxation properties for imaging that are different from the corresponding properties of the cells/tissue being imaged. Suitable contrast agents include an imageable nucleus (such as ¹⁹F), radionuclides, diamagnetic, paramagnetic, ferromagnetic, superparamagnetic substances, and the like. In a preferred aspect, iron-based or gadilinium-based contrast agents are used. Iron-based agents include iron oxides, ferric iron, ferric ammonium citrate and the like. Gadolinium based contrast agents include diethylenetriaminepentaacetic (gadolinium-DTPA). Manganese paramagnetic substances also can be used. Typical commercial MRI contrast agents include Omniscan, Magnevist (Nycomed Salutar, Inc.), and ProHance.

In one preferred aspect, gadolinium is used as a contrast agent. Less than about 28.14 mg/mL gadolinium (such as less than 6% Magnevist) is an adequate concentration for imaging and is minimally destructive of nucleic acid delivery vehicles. However, it should be obvious to those of skill in the art that amounts of contrast agents may be varied and optimized depending on the nature of the contrast agent (e.g., their osmotic effects) and the length of time during which a target cell is exposed.

### Carriers

In one aspect, the composition comprises a pharmaceutically acceptable carrier.. Preferably, the carrier is non-toxic, isotonic, hypotonic or weakly hypertonic and has a relatively low ionic strength (e.g., such as a sucrose solution). Furthermore, it may contain any relevant solvents, aqueous or partly aqueous liquid carriers comprising sterile, pyrogen-free water, dispersion media, coatings, and equivalents, or diluents (e.g. Tris-HCI, acetate, phosphate), emulsifiers, solubilizers and/or adjuvants. The pH of the pharmaceutical preparation is suitably adjusted and buffered in order to be appropriate for use in humans or animals. Representative examples of carriers or diluents for an injectable-composition include water or isotonic saline solutions which are preferably buffered at a physiological pH (e.g., such as phosphate buffered saline, Tris buffered saline, mannitol, dextrose, glycerol containing or not polypeptides or proteins such as human serum albumin).

### Accessory Molecules

The compositions according to the invention may comprise one or more accessory molecules for facilitating the introduction of a nucleic acid delivery vector into a cell and/or for enhancing a particular therapeutic effect. In one preferred aspect, an accessory molecule which is an angiogenic factor is provided.

Suitable angiogenic factors include, but are not limited to: a vascular endothelial growth factor isoforms or family members (e.g., such as VEGF-A₁₂₁, VEGF-A₁₆₅, VEGF-A₁₈₉, and VEGF-A₂₀₆; mouse VEGF-A; VEGF-B; VRF; VEGF-C; VEGF-D; VEGF-E; VRP) (see, e.g., Leung, et al., 1989, Science 246: 1306-1309; U.S. Pat. No. 5,194,596; U.S. Pat. No. 5,240,848; U.S. Pat. No. 5,332,671; Grimmond, et al., 1996, Genome Res. 6:124-131; Lee, et al., 1996,Proc. Natl. Acad. Sci. USA 93:1988-1992; Ogawa, S. et al., 1998, J. Biol. Chem. 273(47): 31273-31282), fibroblast growth factor family members (see, e.g., Goncalves, 1998, Rev. Port. Cardiol. 17 Suppl 2:II11-20); FIGF (see, e.g., Orlandini, et al., 1996, Proc. Natl. Acad. Sci. USA 93: 11675-11680; placenta growth factor (PIGF) (see, e.g., Maglione, et al., 1991, Proc. Natl. Acad. Sci. USA 88: 9267-9271); acidic FGF (aFGF or FGF-1); basic FGF (FGFR-1, FGFR-2, FGFR-3 and FGFR-4); members of the angiopoietin protein family (see, e.g., Davis, 1997, Curr. Top. Microbiol. Immunol. 237: 173-85); Transforming Growth Factor (particularly, Transforming Growth Factor-Beta), Platelet-derived Endothelial Cell Growth Factor Generally, an angiogenic factor is any substance that initiates and/or enhances angiogenesis or neovascularization.

Angiogenic factor activity can be assessed by counting vessels in tissue sections, e.g., following staining for marker molecules (e.g., such as CD3H, Factor VIII or PECAM-1). Other systems that can be used for assessing angiogenic factor activity include an endothelial cell chemotaxis assay. An angiogenic factor or agent can be identified in such an assay by its ability to promote endothelial cell chemotaxis above control values. Other bioassays include the chick CAM assay, the mouse corneal assay, and assays to monitor the effects of administering isolated or synthesized proteins on implanted tumors. The chick CAM assay is described by O'Reilly, et al., 1994, Cell 79: 315-328.

In addition, the composition according to the present invention may include one or more stabilizing substance(s), such as lipids, nuclease inhibitors, hydrogels, hyaluronidase (WO 98/53853), collagenase, polymers, chelating agents (EP 890362), in order to inhibit degradation within the animal/human body and/or improve transfection/infection of the vector into a target cell. Such substances may be used alone or in combination (e.g., cationic and neutral lipids). In one preferred aspect, a carrier comprises one or more substances to facilitate gene transfer in arterial cells, such as a gel complex of poly-lysine and lactose (see, e.g., Nfidoux, et al., 1993, Nucleic Acid Res. 21: 871-878) or poloxamer 407 (Pastore, 1994, Circulation 90: 1-517).

It has also been shown that adenovirus proteins are capable of destabilizing endosomes and enhancing the uptake of DNA into cells. The mixture of adenoviruses to solutions containing a lipid-complexed DNA vector or the binding of DNA to polylysine covalently attached to adenoviruses using protein cross-linking agents may substantially improve the uptake and expression of a nucleic acid delivery vector (see, e.g., Curiel, et al., 1992, Am. I. Respir. Cell. Mol. Biol. 6: 247-252).

Other accessory molecules including drugs, therapeutic agents, peptides, polypeptides, proteins, nucleic acids, small molecules, antibiotics, chemotherapy reagents, toxins, and the like, also may be included in the compositions according to the invention.

In one preferred aspect, the compositions described above are used in vascular gene therapy. For this application, the type of delivery vehicle may be selected which is optimal for the delivery and/or expression of a particular type of gene. Exemplary, but non-limiting combinations are provided below in Table 1.

TABLE 1
Important Vectors And Genes Used In Vascular Gene Therapy

| Treatment | Vector | Gene |
|---|---|---|
| Thrombosis: | Adenovirus | Thrombomodulin, Hirudin |
| | Retrovirus | t-PA, a-UPA |
| Restenosis: | Adenovirus | Thymidine kinase, Cytosine deaminase, |
| | | Retinoblastoma gene product, P21 |
| | | cycling-dependent kinase inhibitor 1, |
| | | ras transdominant mutant, Cyclo- |
| | | oxygenase, TIMP-1, TIMP-2 |
| | Retrovirus | Cyclin GI |
| | Liposome- | Cell cycle regulatory gene, PCNA |
| | Sendai-virus | c-Myc, c-Myb, cdc-2, Nitric oxide |
| | construct | synthase, VEGF, HGF |
| Angiogenesis: | Naked-plasmid | VEGF |

t-PA = tissue plasminogen activator; a-UPA = anchored urokinase-type plasminogen activator; TIMP = tissue inhibitor of metalloproteinase; PCNA = proliferating cell nuclear antigen; VEGF = vascular endothelial growth factor; HGF = hepatocyte growth factor

### Delivery

Methods for delivering the compositions according to the invention to a target cell will vary depending on the type of delivery vehicle being used and the type of treatment being administered. Modes of administration including systemic, enteral, parental, and localized administration. For systemic administration, the compositions can be injected or ingested. Injection may be subcutaneous, intravenous, intraperitoneal, intrathecal, intracardiac (such as transendocardial and pericardial), intramuscular, intratumoral, intrapulmonary, intratracheal, intracoronary or intracerebroventricular and preferably, intravascular or intraarterial. Administration may take place in a single dose or a dose repeated one or several times after a certain time interval. The appropriate administration route and dosage may vary in accordance with various parameters, as for example, the condition or disease to be treated, the stage to which it has progressed, the need for prevention or therapy and the therapeutic nucleic acid (e.g., gene) to be transferred. As an indication, a composition based on viral particles may be formulated in the form of doses of between about 10 and 10¹⁴ i.u., preferably, between about 10 and 10¹¹ i.u., and more preferably, between about 10′ and 10″ iu. The titer may be determined by conventional techniques. The doses of vector are preferably comprised between 0.01 and 10 mg/kg, more preferably, between 0.1 and 2 mg/kg.

Targeted cells also can vary depending on the treatment application, and include, but are not limited to, cells of the heart, liver, prostate, breasts, kidneys, brain, thyroid, and muscles.

In a particularly preferred aspect, the nucleic acid delivery vehicle is delivered to a target cell through a medical access device 1, such as a catheter (e.g., an angiographic catheter, embolization catheter, perfusion catheter, and gene/drug delivery catheter, and the like). In one aspect, the medical access device comprises a housing 2 defining at least one lumen 3, the housing conforming in shape to a body cavity or lumen, such as a blood vessel. Generally, the housing is substantially tubular in shape along at least a portion of its length sufficient to allow the housing to be inserted and navigated within the body cavity or lumen. In one aspect, the housing comprises a first end and a second end. Preferably, the first end comprises a sheath (not shown) which surrounds the housing 2 and a dilation balloon 4 compressed to the diameter of the sheath during navigation of the device 1. In operation, the dilation balloon 4 can be inflated by being filled with water, saline, contrast agent, or optically transparent solutions or fluids.

Balloons used in medical access devices are well known and, thus, although described and shown with reference to a preferred embodiment, the general features (e.g. size, shape, materials) of the dilation balloon 4 may be in accordance with conventional balloons. In a preferred embodiment, the balloon 4 is made of a biocompatible, distendable material (e.g., including, but not limited to, flexible medical-grade silicone rubber or polyethylene terepthalate (PET)) which is capable of being inflated to a size sufficient to compress a target lumen's walls. It should be obvious to those of skill in the art, therefore, that the exact dimensions of the balloon should be configured to the type of lumen/vessel/cavity being accessed.

The medical access device 1 may comprise multiple lumens or channels for increasing the functionality of the device 1. Multiple channels may be concentric or coaxial, may share walls or comprise separate walls. The multiple channels also may converge at one or more points along the length of the housing 2. These features are well known in the art of medical device design. In one preferred aspect, the device 1 comprises a delivery channel 5 for delivering the gene delivery compositions according to the invention to a target cell. The housing also may comprise a guidewire channel 6 for insertion of a guidewire to assist in navigation of the device 1. Preferably, the device 1, may additionally, or alternatively, comprise a channel for placement of one or more optical fibers (not shown), to aid in imaging of the body cavity/lumen/vessel. For example, the optical fiber(s) can be used to receive fluorescent light from cells which have incorporated a vector comprising a fluorescent reporter gene as described above. The optical fibers also may be used to monitor the navigation of the device itself. To this latter end, the surface of the housing 2 (e.g., closer to the walls of the body cavity/lumen/vessel) may be marked with one or more radioopaque markers. In still other aspects, a channel may be provided for accepting an ultrasonic probe for providing treatment to a target tissue in the form of ultrasound, which may be used to complement nucleic acid delivery treatment methods.

Most preferably, the device 1 comprises an inflation channel (not shown) comprising at least one exit port (not shown) with an opening which communicates with the dilation balloon to deliver an inflating fluid to the dilation balloon, thereby inflating the balloon. The inflation pressure in the delivery balloon can be maintained at a constant value using an infusion pump (erg., such as the Harvard Apparatus, Holliston, Mass.). Preferably, the dilation balloon 4 comprises one or more perfusion channels 8 to allow a biological fluid, such as blood, to flow into the distal portion of a cavity/lumen/vessel being accessed which is otherwise blocked by the device 1. More preferably, the dilation balloon comprises a plurality of perfusion channels 8, about 100 to about 500 μm in diameter.

This design increases the time that the inflated balloon can remain in contact with the walls of a lumen, e.g., such as a blood vessel. In an intravascular gene delivery, the time during which a composition according to the invention can be administered is generally increased because blood flow through the perfusion channels allows the inflated balloon to remain within the target vessel for a longer period of time, minimizing distal thrombosis, and increasing the efficacy of gene delivery.

In another aspect, the device 1 comprises a second balloon, or a delivery balloon 7, which communicates with at least one exit port (not shown) of the delivery channel 5 and which inflates when a fluid from the delivery channel 5 (e.g., comprising a composition described above) flows from the exit port into the delivery balloon 7. The delivery balloon 7 may be closely opposed to the dilation balloon during navigation (i.e., in an uninflated state). Preferably, the delivery balloon 7 is porous, comprising a plurality of microholes. (The term “microhole” implies no particular limitation on size). In one aspect, a plurality of linearly-arrayed, 15-25 μm microholes are disposed on at least one lateral surface of the delivery balloon 7.

During vascular interventional procedures, the dilation balloon 4 may cause intimal tears and subintimal dissection (see, e.g., Zollikofer, et al., 1992, In Interventional Radiology, W. Castaneda-Zuniga and S. Tadavarthy, Editors, Williams & Wilkins: Baltimore, Md., pp. 249-297). This is exploited in the design of the porous delivery balloon which essentially directly injects compositions from the delivery channel (e.g., contrast agents and gene delivery vehicles) to these areas. The effects of balloon inflation “injury” and accumulation of the contrast agents at these areas results in contrast enhancement of the target vessel wall, which is visualized during high-resolution MRI. Infusion may be enhanced by providing needles or other penetrating elements on the surface of the device 1.

Methods for navigating catheters to desired target locations are well known in the art and described in, for example, Rutherford, Vascular Surgery, P edition (Saunders Co 1989). In one aspect, the device is used to deliver a gene delivery vehicle: contrast agent mixture to vessels in the vicinity of a stenosis or an area of ischemia.

### Imaging

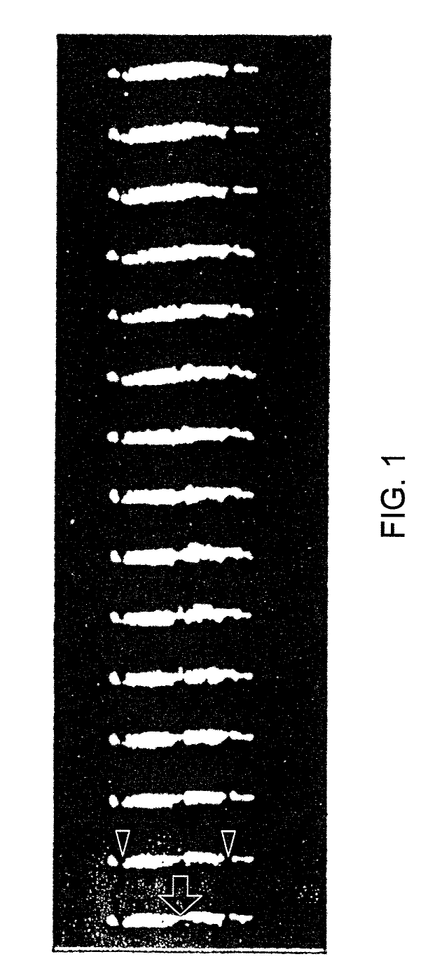

MRI has two particular advantages over other techniques: high spatial resolution and tissue contrast that simultaneously allow acquisition of physiologic and anatomic information (Johnason, et al., 1993, supra; Weissleder, et al., 1997, Radiology 204: 425-429). MRI allows for high-resolution images of a blood vessel (including the vessel wall); multiple diagnostic evaluations of organ function and morphology; and multiple image planes with no risk of ionizing radiation. MRI is commonly used to monitor balloon angioplasty procedures (see, e.g., as shown in FIG. 1).

Molecular MRI also has also been used to monitor cell trafficking (see, e.g., Dodd, et al., 1999, Biopys. J. 76: 103-109). Currently, MRI is widely used to aid in the diagnosis of many medical disorders. (see, for example, 1993, Edelman & Warach, Medical Progress 328:708-716 (1993); Edelman and Warach, 1993, New England J. of Medicine 328: 785-791).

Magnetic resonance imaging techniques are described, for example, D. M. Kean and M. A. Smith, Magnetic Resonance Imaging: Principles and Applications, (Williams and Wilkins, Baltimore 1986). Suitable MRI techniques include, but are not limited to, nuclear magnetic resonance (NMR) and electronic spin resonance (ESR). In one aspect, NMR is performed.

Nuclei with the appropriate nuclear spin align in the direction of an applied magnetic field. The nuclear spin may be aligned in either of two ways: with or against the external magnetic field. Alignment with the field is more stable; while energy must be absorbed to align in the less stable state (i.e., against the applied field). In the case of protons, these nuclei resonate at a frequency of 42.6 MHz in the presence of a 1 tesla (1 tesla=10⁴ gauss) magnetic field. At this frequency, a radio-frequency (RF) pulse of radiation will excite the nuclei and change their spin orientation to be aligned against the applied magnetic field. After an RF pulse, the excited nuclei “relax” or return to equilibrium or in alignment with the magnetic field. The decay of the relaxation signal can be described using two relaxation terms. T1, the spin-lattice relaxation time or longitudinal relaxation time, is the time required by the nuclei to return to equilibrium along the direction of the externally applied magnetic field. The second, T2, or spin-spin relaxation time, is associated with the dephasing of the initially coherent precession of individual proton spins. The relaxation times for various fluids, organs and tissues in different species of mammals is well documented.

One advantage of MRI is that different scanning planes and slice thicknesses of tissues can be selected and imaged without loss of resolution. This permits high quality transverse, coronal and sagittal images to be obtained directly. The absence of any mechanical moving parts in the MRI equipment promotes a high degree of reliability. It is generally believed that MRI has greater potential than X-ray computer tomography (CT) for the selective examination of tissues.

Due to subtle physio-chemical differences among organs and tissue, MRI may be capable of differentiating tissue types and in detecting diseases that may not be detected by X-ray or CT. In comparison, CT and X-ray are only sensitive to differences in electron densities in tissues and organs. The images obtainable by MRI techniques can also enable a physician to detect structures smaller than those detectable by CT, due to its better spatial resolution. Additionally, any imaging scan plane can be readily obtained using MRI techniques, including transverse, coronal and sagittal.

An MRI system typically includes an imaging coil and a platform for supporting a subject in a substantially horizontal posture. Preferably, the platform can move with respect to the imaging coil. The system also includes imaging device or detector for collecting image data of the subject while at each position of the platform. Movement of the platform may be coordinated with image acquisition so that the platform moves only after an image is acquired. Therefore, in one preferred aspect, the imaging device and platform are in communication with a processor for receiving input from the imaging device and providing output to the platform and visa versa. Preferably, the processor is in communication with a work station comprising a computer and display monitor and displays images to a user of the system. In one aspect, movement of the platform and various imaging parameters is controlled by the user.

The processor and imaging coil apparatus may be a commercial magnetic resonance imaging system (including hardware and software). For example, General Electric's Horizon system, Siemens' Vision system, or Phillips' Gyroscan system can be used. These imaging systems are suitable for imaging an animal body, for example, a transgenic animal or animal to be made transgenic, or human, and system software can be modified to suit a user's preferences.

Imaging of the nucleic acid delivery vehicles generally involves initially irradiating a subject placed in a uniform magnetic field with radiation, usually VHF radiation, of a frequency selected to excite a transition in a contrast agent administered to, the subject along with the nucleic acid delivery vehicle. Dynamic nuclear polarization results in an increase in differences between the excited and ground nuclear spin states of populations of selected nuclei, i.e., those nuclei, generally protons, which are responsible for the magnetic resonance signals (MR imaging nuclei). MR signal intensity is proportional to this population difference.

Measurements are preferably carried out in a way that maximizes the Contrast-to-Noise-Ratio (CNR), defined as the signal change during administration of the composition divided by the noise. For a given contrast agent, the CNR will depend on the Echo Time (TE) of the MRI sequence and on the concentration of the agent in blood (and therefore on the administered dose). Longer echo times will increase the signal change during administration but will also increase the noise in baseline post-contrast scans. The same effects are obtained increasing the concentration of the contrast agent. The optimum signal drop from pre-contrast to baseline post-contrast scans can be computed and optimized using methods well known in the art.

Administration of a composition according to the invention to a selected region of a subject, e.g., by injection or by using the medical access device 1, described above, means that the contrast effect may be localized to a region in proximity to the site of injection or to the medical access device 1. The precise effect depends on the extent of biodistribution of the composition over the period in which the contrast agent remains significantly polarized.

In one aspect, the patient is secured to the platform and the platform is positioned in a first location. Prior to the administration of a nucleic acid delivery vehicle:magnetic resonance contrast agent mixture, the imaging system applies a series of magnetic resonance pulses (radio frequency pulses) to a first region of interest in the patient. The detection system measures or determines a baseline or pre-contrast response of the region of interest (artery and/or tissues in the region of interest) to that series of pulses. The series of magnetic resonance pulses are applied to the patient to tip the longitudinal magnetization of protons in the region of interest and to measure the response of the region of interest before administration of the contrast agent to the patient. The response signal from the region of interest is monitored using a variety of coils of the imaging coil apparatus and is measured by the detection system.

After a baseline or pre-contrast response is measured, the contrast agent may be administered to the patient. Thereafter, the detection system measures (continuously, periodically or intermittently) the response from the region of interest to detect the “arrival” of the contrast agent in the region of interest. The magnetic MRI system applies a series of magnetic resonance pulses and the detection system evaluates the response from the region of interest. When contrast agent “arrives” in the region of interest (e.g., such as an artery or arteries of interest), the detection system detects a characteristic change in the response from the region of interest to the magnetic resonance pulses; i.e., a change in the radio frequency signal emitted from the region of interest. This characteristic change in radio frequency signal from the region of interest indicates that the contrast agent has “arrived” in target region. The detector relays signal to the processor which initiates the process of data collection until an image is generated. However, in other embodiments, the processor collects data at predetermined intervals.

In a particularly preferred aspect, an intravascular magnetic resonance imaging (MRI) technique is used which involves inserting a novel loopless antenna into vessels (Ocali and Atalar, 1997, MRM 37:112-118). Using this technique, high-resolution MR images of arterial walls and atherosclerotic plaques can be obtained. The acquisition of real-time MR fluoroscopic images can be used to guide intravascular interventions (see, e.g., Correia, et al., 1997, Arterioscler. Thromb. Vasc. Biol. 17: 3626-2632; Yang and Atalar, 1999, Circulation 100: 1-799; Yang and Atalar, 2000, Radiology 217: 501-506; Yang, et al., 2001, Circulation 104: 1588-1590.

As discussed above, other imaging modalities besides MRI can be used, such as CT, X-ray, and the like. Methods of implementing these techniques are routine in the art and encompassed within the scope of the invention.

### EXAMPLES

The invention will now be further illustrated with reference to the following examples. It will be appreciated that what follows is by way of example only and that modifications to detail may be made while still falling within the scope of the invention.

### Example 1

### Admixture of Gene-Carrying Viral Vector and MRI Contrast Agents

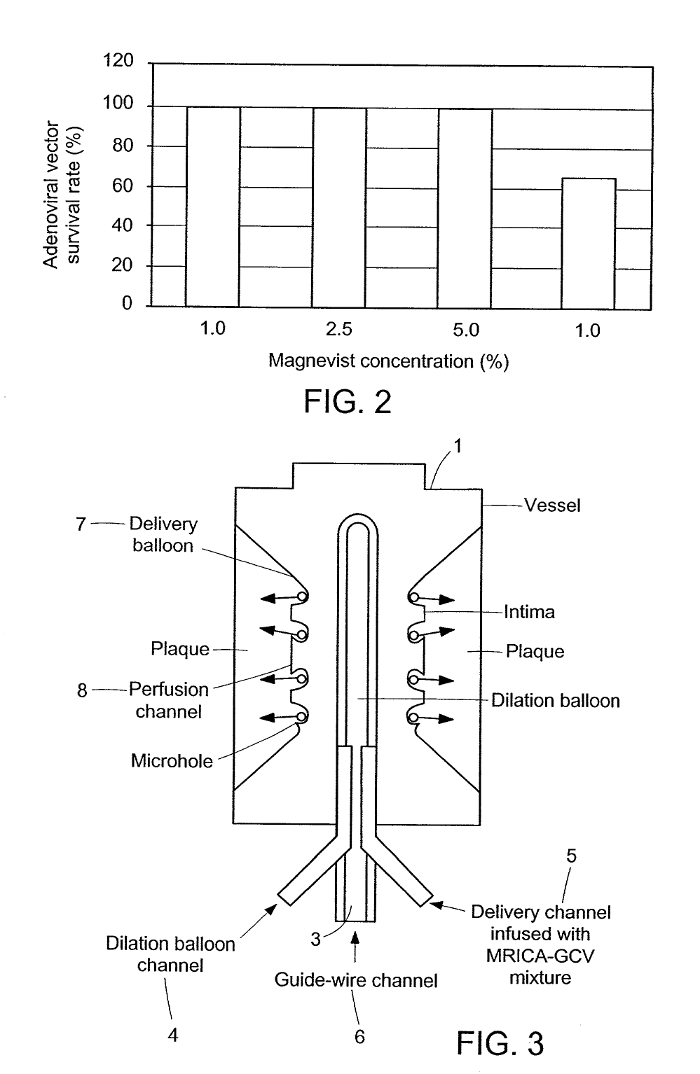

As shown in FIG. 2, good adenoviral vector survival is observed when adenoviral vectors are mixed with different concentrations of Magnevist. With less than 5% Magnevist, 100% survival was observed. 65% survival was observed when 10% Magnevist was used. Thus, ranges of Magnevist from about 10% or less, are generally suitable for gene delivery. A 5-6% Magnevist concentration is optimal for demonstrating balloon inflation under intravascular MR imaging (FIG. 1)

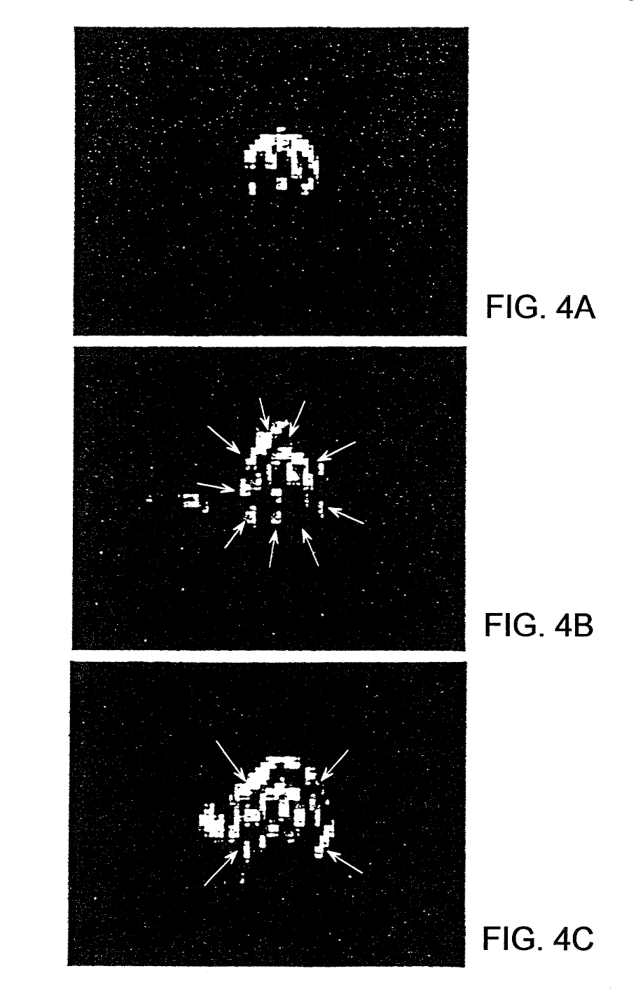

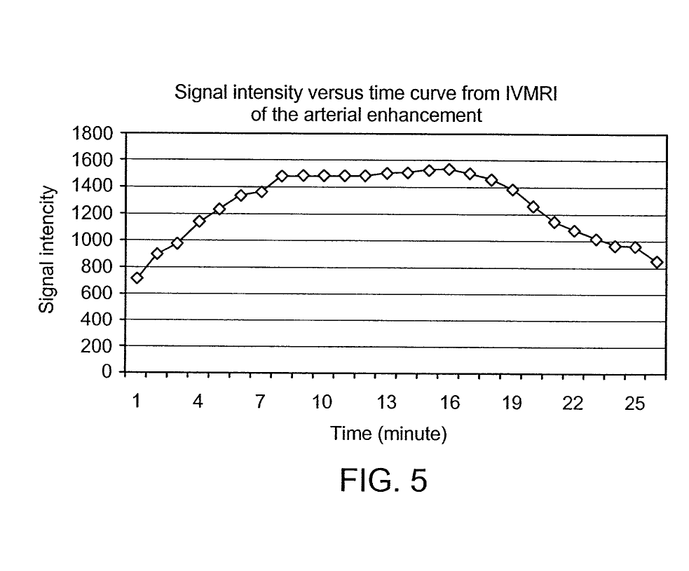

To monitor delivery into a target vessel, the following MR imaging parameters were used: ECG-gated FSE pulse sequence; 1700/88-msec TRITE; 90° flip angle; 15.6 kHz; 8×8 FOV; 256×128 matrix; and 3-mm slice thickness with no spacing (FIGS. 4A-C and FIG. 5). Since some intracellular contrast agent can remain within target cells for several days (Young, et al, 1994, Investigative Radiology 29: 330-338; Young and Fan, 1996, Investigative Radiology 3/:280-283. 22), the immediate distribution of the gene-vector can be tracked by visualizing the MRI-contrast agent-enhancement location within the target vessel wall.

A gene-vector solution can be mixed with a contrast agent to produce an optimum gene vector-contrast agent medium, which has the highest gene-vector transfection capability and the best MRI enhancement of a target vessel wall. The gene-vector transfection capability is quantified using routine laboratory techniques, such as immunohistochemistry; quantitative flow cytometric analysis, and western blot analysis, while the signal intensity of the optimum gene-vector/ contrast agent medium is evaluated using T1- and T2-weighted MR images with different pulse sequences.

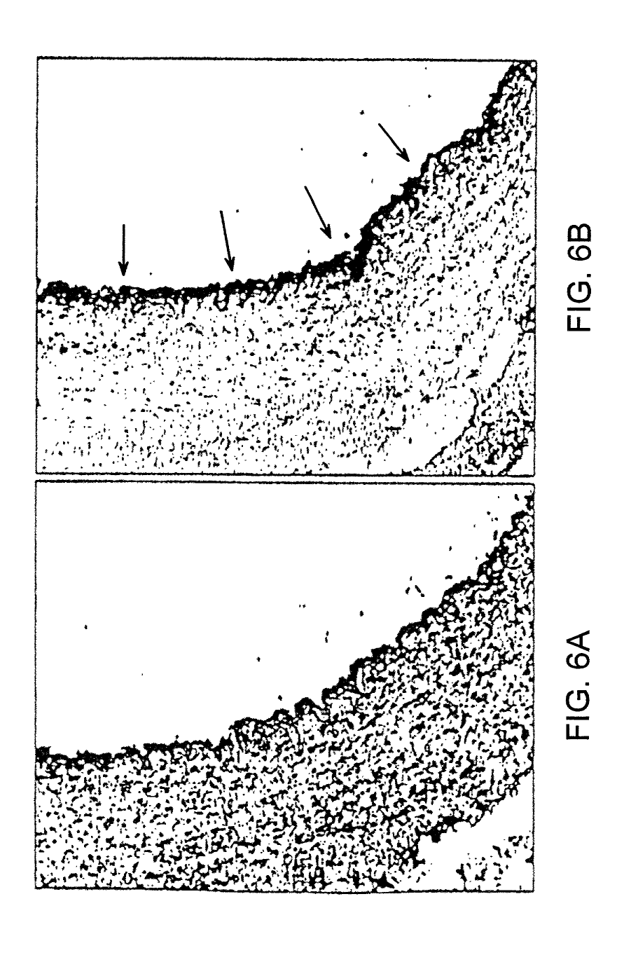

Vectors can be constructed with both marker genes, such as fluorescent protein genes, and therapeutic genes. In one instance, after a mixture of nucleic acid delivery vector and contrast agent was administered to an animal subject (e.g., such as a pig, rabbit, or rat), the animal was kept alive for several days to to allow fluorescent gene expression. Then, targeted arterial portions were harvested and cut into two pieces: one for immunohistochemistry confirmation (FIGS. 6A and B) and one for either quantitative flow cytometric analysis or Western blot analysis (not shown).

### Example 2

### In Vivo MRI of Vascular Gene Delivery

This study was divided into two sections: in vitro and in vivo. For in vitro experiments, an 8-mm homemade porous balloon catheter was inserted into a 6-mm fresh cadaver human iliac artery segment, and the porous balloon was inflated with 6% Magnevist for 10 minutes. Axial MR images of the artery were taken before and after the gadolinium inflation, using a fast spin-echo (FSE) sequence, 2000/16-msec TR/TE, 8-cm FOV, 256 matrix, and 3-mm thickness. Then, with a phantom, a Remedy gene delivery balloon catheter (channel catheter, Scimed, Boston) was used to confirm that the composition could be imaged using an existing, tested catheter system implementing balloon inflation and channel infusion under MR imaging.

For in vivo experiments, a 3.5- or 4.0-mm Remedy balloon catheter was position into either the iliac arteries or the femoral arteries (3.0- or 3.5-mm in diameter) of seven 20 to 25-kg domestic pigs under X-ray fluoroscopy guidance (FIGS. 7A-C). In four pigs, 6% Magnevist mixed with trypan-blue was delivered into the target arterial wall at a balloon inflation pressure of 3.0 Atm and an infusion rate of 6.5 mL/hour for 20 minutes. The gadolinium was used as a marker for MR imaging and the blue-dye as a marker for histopathological examination. By combining a 0.014″ MR imaging-guidewire (Surgi-vision, Inc. Gaithersburg, Md.) with the Remedy balloon catheter, the gadolinium/blue delivery procedure was monitored under intravascular MR imaging using an ECG-gated FSE sequence, 3000/64-msec TR/TE, 62.5 kHz, 8×8 FOV, 90° flip angle, and 3-mm thickness.

In two pigs, delivery of green fluorescent protein (GFP)-lentiviral vectors into target arteries was tested with balloon inflation at 3.0 Atm and GFP-lentiviral infusion at 6.5 mL/hour for 30 minutes (see, e.g., Nabel, 1995, supra). Subsequently, in the remaining pig, a GFP-lentivirus/Magnevist mixture (with a net concentration of gadolinium at 6%) was delivered into the target artery using the same experimental and MRI protocols as described above. In all in vivo experiments, unilateral target arteries were either infused with Magnevist/blue-dye or transfected with GFP-lentivirus only or GFP-lentivirus/Magnevist mixture, while the opposite corresponding arteries were neither infused nor transfected to serve as controls.

In the pigs infused with Magnevistiblue dye, the target vessel was immediately harvested for histopathology examination to confirm the success of the transfer. For the pigs transfected with GFP-lentiviral vector only, or with the GFP-lentivirus/Magnevist mixture, the pig was kept alive for five days to allow sufficient GFP expression. Then, at day six, pigs were euthanized and the bilateral target arteries were harvested to assess the success of the transfection using immunohistochemistry.

The in vitro experiment with the human cadaver artery showed clearly the signal intensity increase of the entire arterial wall immediately after gadolinium delivery. In the pigs infused with Magnevist/blue dye or transfected with GFP-lentivirus/Magnevist, the gadolinium enhancement of the target arterial wall under intravascular MR imaging (FIG. 8) could be dynamically visualized. The success of gene transfer was confirmed by histopathology and immunohistochemistry (FIGS. 9A and B).

The gadolinium enhancement of the vessel wall is most likely initiated by the balloon over-inflation that causes tears in the intima with consequent dehiscence of this layer from the media (Zollikofer, et al., In Interventional Radiology, Castaneda-Zuniga. W., and Tadavarthy, S., ed., 1992, Williams & Wilkins: Baltimore, Md., p249-297). Thus, the GFP-lentivirus/gadolinium medium can enter the target vessel wall through the lateral pores of the balloon and the torn intima, and remain in the dehiscence. Using the Remedy gene delivery balloon catheter, both GFP-lentivirus vectors and gadolinium could be successfully delivered into the vessel wall, and could be monitored using intravascular high-resolution MR imaging.

Variations, modifications, and other implementations of what is described herein will occur to those of ordinary skill in the art without departing from the spirit and scope of the invention and claims herein. All patents, patent publications, international applications, and references are incorporated by reference herein in their entireties.

What is claimed is:

1. A composition comprising an admixture of a nucleic acid molecule and a contrast agent.

2. The composition according to claim 1, wherein the nucleic acid molecule comprises DNA, RNA, an antisense molecule, a ribozyme, an oligonucleotide, an aptamer, or a modified form thereof.

3. The composition according to claim 1, wherein the nucleic acid molecule comprises a nucleic acid delivery vector.

4. The composition according to claim 2, wherein the vector comprises a plasmid, an adenoviral vector, retroviral vector or an adeno-associated viral vector.

5. The composition according to claim 1, wherein the nucleic acid molecule is provided in a nucleic acid delivery vehicle.

6. The composition according to claim 5, wherein the delivery vehicle is lipid-based, viral-based, or cell-based.

7. The composition according to claim 5, wherein the delivery vehicle comprises a multilamellar liposome, a gas-filled microbubble or a fluorocarbon emulsion.

8. The composition according to claim 3, wherein the vector comprises a gene operably linked to an expression control sequence.

9. The composition according to claim 3 or 8, wherein the vector comprises a marker gene.

10. The composition according to claim 9, wherein the marker gene is a fluorescent protein.

11. The composition according to claim 1, wherein the contrast agent is a magnetic resonance imaging contrast agent.

12. The composition according to claim 11, wherein the composition comprises iron or gadolinium

13. The composition according to claim 1, wherein the nucleic acid molecule comprises nucleic acids comprising at least two different genes.

14. The composition according to claim 1, further comprising an agent selected from the group consisting of: a drug, an angiogenic factor, a growth factor, a chemotherapeutic agent, a radionuclide, a protein, a polypeptide, a peptide, a viral protein, a lipid, an amphiphile, a nuclease inhibitor, a polymer, a toxin, a cell, and modified forms, and combinations thereof.

15. The composition according to claim 1, wherein the nucleic acid molecule comprises a sequence encoding a polypeptide selected from the group consisting of hirudin, tissue plasminogen activator, an anchored urokinase activator, a tissue inhibitor of metalloproteinase, proliferating cell nuclear antigen, an angiogenic factor, a tumor suppressor, a suicide gene and a neurotransmitter.

16. A medical access device, comprising:
a housing defining a plurality of channels, at least one channel comprising a delivery channel comprising at least one exit port and at least one channel comprising an inflation channel comprising at least one exit port;
a dilation balloon in communication with the at least on exit port of the inflation channel, the dilation balloon comprising at least one perfusion channel;
a delivery balloon in communication with the at least one exit port of the delivery channel; the delivery balloon comprising a plurality of pores.

17. The medical access device of claim 16, wherein at least one channel is selected from the group consisting of: a guidewire channel, a channel for an optical probe, and a channel for an ultrasound probe.

18. The medical access device of claim 16, wherein the device is a catheter.

19. The medical access device of claim 18, wherein the catheter is selected from the group consisting of an angiographic catheter, an embolization catheter, a perfusion catheter, and delivery catheter.

20. A method for delivering a nucleic acid to a target cell comprising administering the composition of claim I to the target cell.

21. The method of claim 20, wherein the target cell is selected from the group consisting of a heart cell, liver cell, prostate cell, kidney cell, neural cell, thyroid cell, muscle cell, hematopoietic cell, circulating cell, a cell of a blood vessel, and a neoplastic cell.

22. The method according to claim 20, wherein the target cell is part of a multicellular organism.

23. The method according to claim 20, further comprising detecting a signal associated with the contrast agent.

24. The method according to claim 23, wherein the signal comprises a magnetic resonance signal.

25. The method according to claim 20, further comprising localizing the signal to a location in the multicellular organism.

26. The method according to claim 25, wherein localizing the signal to the location indicates delivery of the nucleic acid molecule to the location.

27. The method according to claim 20, wherein the nucleic acid encodes a gene product necessary for correcting, normalizing, and/or preventing an abnormal physiological response by the target cell.

28. The method according to claim 20, wherein the nucleic acid molecule further comprises a marker gene and the presence of the marker gene in the target cell is determined.

29. The method according to claim 28, wherein the expression of the marker gene is determined.

30. The method according to claim 20, wherein the nucleic acid molecule encodes a gene selected from the group consisting of hirudin, tissue plasminogen activator, an anchored urokinase activator, a tissue inhibitor of metalloproteinase, proliferating cell nuclear antigen, an angiogenic factor, a tumor suppressor, a suicide gene and a neurotransmitter.

31. The method according to claim 20, wherein the nucleic acid molecule is encapsulated within a viral capsid.

32. A method for delivering an agent to a target cell, the method comprising:
positioning a medical access device according to claim 16 in the lumen of a body vessel comprising the target cell or which perfuses a tissue comprising the target cell;
inflating the dilation balloon to compress the walls of the blood vessel, while permitting bodily fluids to flow through the lumen through at least one perfusion channel of the dilation balloon;
delivering a solution comprising the agent through the delivery channel to the delivery balloon and from the delivery balloon to at least a portion of an inner wall of the body lumen, through the plurality of pores in the delivery balloon.

33. The method according to claim 32, wherein the target cell is an endothelial cell.

34. The method according to claim 32, further comprising monitoring delivery of the agent by detecting a signal associated with a contrast molecule.

35. The method according to claim 32, further comprising imaging the body vessel.

36. The method according to claim 32, further comprising imaging navigation of the device in the body vessel.

37. The method according to claim 32, wherein the agent comprises an admixture of a nucleic acid molecule and a contrast agent.
